(19) United States
(12) Patent Application Publication
(10) Pub. No.: US 2026/0232922 A1
(43) Pub. Date: Aug. 13, 2026

(54) A MEDICAMENT DELIVERY DEVICE

(71) Applicant: SHL Medical AG, Zug (CH)

(72) Inventor: Daniel Scott, Deerfield Beach, FL (US)

(21) Appl. No.: 19/154,162

(22) PCT Filed: Jan. 30, 2024
(86) PCT No.: PCT/EP2024/052250

§ 371 Date: Aug. 6, 2025

Related U.S. Application Data
(60) Provisional application No. 63/444,599, filed on Feb. 10, 2023.
(60) Provisional application No. 63/527,095, filed on Jul. 17, 2023.

(51) Int. Cl. A61M 5/32

(52) U.S. Cl. CPC A61M 5/3245; A61M 2005/3267; A61M 2205/581; A61M 2205/582

(57) ABSTRACT

The present disclosure provides a medicament delivery device. The medicament delivery device includes a housing having a proximal end and a distal end. The medicament delivery device further includes a medicament container arranged within the housing and a plunger rod movably arranged within the housing. The plunger rod is configured to be moved into the medicament container so that contained medicament can be expelled. The plunger rod comprises an interaction member. The medicament delivery device further includes a clicking ring arranged at a distal end of the medicament container. The counter interaction member of the clicking ring configured to interact with the interaction member of the plunger rod when the plunger rod is moved relative to the clicking ring such that audible/tactile feedback is generated to indicate a progress of expelling the medicament The clicking ring configured to rattle on the medicament container when the counter interaction member of the clicking ring interacts with the interaction member of the plunger rod.

### TECHNICAL FIELD

The present invention relates to a medicament delivery device and more particularly to the medicament delivery device with automatic functions.

### BACKGROUND

Medicament delivery devices such as auto-injectors, inhalers, or on-body devices are generally known for the self-administration of a medicament by patients without formal medical training. For example, patients suffering from diabetes or people who are undergoing an artificial fertilization procedure may require repeated injections of insulin or hormone. Other patients may require regular injections of other types of medicaments, such as a growth hormone. Therefore, medicament delivery devices for self-administration usually are arranged with multiple automatic functions and protection features. For example, a medicament delivery member guard is commonly used. The medicament delivery member guard is configured to cover a medicament delivery member, e.g., an injection needle, so that a user will not accidentally be in contact with the medicament delivery member.

The medicament delivery member guard is usually arranged to be telescopically movable relative to the housing of the medicament delivery device. The medicament delivery member guard is configured to be in contact with the medicament delivery site. A medicament delivery device that is configured to be triggered, e.g., inserting the injection needle into the medicament delivery site and/or expelling contained medicament to the medicament delivery site, by the movement of the medicament delivery member guard, for example, the medicament delivery device is triggered once the medicament delivery member guard is fully pressed against the medicament delivery site, or the medicament delivery device is triggered when the medicament delivery member guard is fully pressed against the medicament delivery site and a button is manually pressed by the user is a preferred solution in the market. However, there are still rooms to improve such design.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a medicament delivery device, comprising: a housing having a proximal end and a distal end; a medicament container arranged within the housing; a plunger rod movably arranged within the housing; wherein the plunger rod is configured to be moved into the medicament container so that contained medicament can be expelled; wherein the plunger rod comprises an interaction member; a clicking ring arranged at a distal end of the medicament container; wherein the counter interaction member of the clicking ring configured to interact with the interaction member of the plunger rod when the plunger rod is moved relative to the clicking ring such that audible/tactile feedback is generated to indicate a progress of expelling the medicament; and wherein the clicking ring configured to rattle on the medicament container when the counter interaction member of the clicking ring interacts with the interaction member of the plunger rod.

According to one embodiment, the interaction member of the plunger rod is ratchet teeth extending in the direction of the longitudinal axis.

According to one embodiment, the interaction member of the plunger rod is ratchet teeth extending in the direction of the longitudinal axis.

According to one embodiment, the counter interaction member of the clicking ring is a flexible arm.

According to one embodiment, the medicament delivery device comprises a container carrier.

According to one embodiment, the medicament container is fixed within the container carrier.

According to one embodiment, the clicking ring is attached to a distal end of the container carrier.

According to one embodiment, the clicking ring is loosely attached to a distal end of the container carrier.

According to one embodiment, the clicking ring comprises a proximally directed hook configured to engage with a recess/cut-out of the container carrier such that the clicking ring is attached to the container carrier.

According to one embodiment, the hook is movable within the recess/cut-out.

According to one embodiment, a delivery member cover arranged within the housing and is movable relative to the housing; a tubular rotator releasably engaged with the plunger rod.

According to one embodiment, the tubular rotator is engaged with the delivery member cover and is configured to be rotated by the delivery member cover to release the plunger rod.

According to one embodiment, the tubular rotator comprises a tubular body having at least one retaining member on its inner surface configured to releasably engaged with the plunger rod.

According to one embodiment, the tubular rotator comprises a tubular body having at least one retaining member on its inner surface.

According to one embodiment, the tubular rotator comprises a tubular body having a first and a second retaining member on its inner surface

According to one embodiment, the biased plunger rod comprises a counter retaining element configured to interact with the at least one retaining member or selectively with each of the first, the second and the third retaining members.

According to one embodiment, the tubular rotator comprises a tubular body having a retaining member on its inner surface; and the biased plunger rod comprises a first and a second counter retaining elements, wherein the first and the second counter retaining elements is configured to interact selectively with the retaining member.

According to one embodiment, the tubular rotator comprises a tubular body having a first, a second and a third retaining member on its inner surface; and the biased plunger rod comprises a counter retaining element, wherein the counter retaining element is configured to interact selectively with each of the first, the second and the third retaining members.

According to one embodiment, the retaining member is a radially inward protruding rib.

According to one embodiment, the first and the second retaining members are ledges.

According to one embodiment, the counter retaining member is a radially outward protruding arm.

According to one embodiment, the first and the second counter retaining members are grooves.

According to one embodiment, the medicament delivery device further comprises a medicament container configured to receive the medicament container.

According to one embodiment, the medicament container is axially fixed to the container carrier.

According to one embodiment, the container carrier comprises a dose stop arranged on its distal end.

According to one embodiment, the biased plunger rod further comprises an interaction member configured to interact with a counter interaction member on the distal end of the container carrier.

According to one embodiment, the interaction between the interaction member and the counter interaction member is configured to provide a feedback to a user of the medicament delivery device.

According to one embodiment, the biased delivery member cover comprises a guide element; and the tubular rotator comprises a tubular body arranged with a guide track on its outer surface; wherein the guide element is configured to interact with the guide track such that an axial movement of the biased delivery member cover causes the tubular rotator to rotate.

According to one embodiment, the biased delivery member cover comprises a delivery member cover link; wherein the guide element is arranged on the delivery member cover link.

According to one embodiment, the medicament delivery device is in an initial state when the rotator is in a first rotation position which is defined when the biased plunger rod is in an initial position in which the counter retaining element is engaged with the first retaining member; when the biased delivery member cover is in a first retracted position in which the guide element is positioned on a first distal end point of the guide track; and when the biased delivery member cover is held in said position by the cap assembly which is releasably connected to the housing.

According to one embodiment, the medicament delivery device is in the initial state when the rotator is in a first rotation position which is defined when the biased plunger rod is in an initial position in which the counter retaining element is engaged with the first retaining member; when the biased delivery member cover is in a first retracted position in which the guide element is positioned on a first distal end point of the guide track; and when the biased delivery member cover is held in said position by a knob assembly which axially fixed connected to the distal end of the housing.

According to one embodiment, the medicament delivery device is in a pre-activated state when the rotator is in a second rotation position which is defined when the biased plunger rod is in a pre-activated position in which the counter retaining element is engaged with the second retaining member, and when the biased delivery member cover is in a first extended position in which the guide element is positioned on a first proximal end point of the guide track after the cap assembly is removed from the housing.

According to one embodiment, the medicament delivery device is in the pre-activated state when the rotator is in a second rotation position which is defined when the biased plunger rod is in a pre-activated position in which the counter retaining element is engaged with the second retaining member, and when the biased delivery member cover is in a first extended position in which the guide element is positioned on a first proximal end point of the guide track after the knob assembly is turned from a first knob position to the second knob position.

It should be noted that, the initial state of the medicament delivery device is the state that the medicament delivery device being stored and delivered to the end user, e.g., patients, caregivers, and/or pharmacies; the pre-activated state is the state that the end user puts the medicament delivery device in a ready-to-be-used state but not yet performs the medicament delivery operation, e.g., delivering medicament to a targeted delivery site. The pre-activated state can be defined as a priming state, e.g., the movement of the plunger rod is configured to expel gas within the medicament container; and/or be defined as a mixing state, e.g., the movement of the plunger rod is configured to mix multiple substance with multiple chambers respectively; and/or be defined as a calibrating state, e.g., the movement of the plunger rod moves itself to a ‘dose zero’ position such that the medicament can be delivered to the targeted delivery site is defined between a end of dose stop of the plunger rod and the dose zero position of the plunger rod.

According to one embodiment, the biased plunger rod is configured to move from the pre-activated position to a final position in which the counter retaining element is engaged with the third retaining member, when the biased delivery member cover is moved from the first extended position to a second retracted position in which the guide element is positioned on a second distal end point of the guide track such that the medicament contained in the medicament container is thereby delivered to a user of the medicament delivery device.

It should be noted that, the pre-activated state is optional. In other words, the second retaining member of the rotator is optional. In one example where the rotator does not have the second retaining member, the plunger rod is configured to engage with the first retaining member of the rotator until the medicament delivery device is triggered, in this example, the plunger rod is configured to move from the initial position to the final position in which the counter retaining element is engaged with the third retaining member of the rotator.

According to one embodiment, the biased delivery member cover is axially movable from the first retracted position to the first extended position; from the first extended position to the second retracted position; and from the second retracted position to a second extended position in which the guide element is positioned on a second proximal end point of the guide track; such that the delivery member is extended from the delivery member cover when the delivery member cover is in the first and the second retracted positions; and is covered by the delivery member cover when the delivery member cover is in the first and the second extended positions.

According to one embodiment, the counter retaining element is configured to disengage with the second retaining member when the biased delivery member cover is axially movable from the first extended position to the second retracted position such that the rotator is moved from the second rotation position to a third rotation position.

According to one embodiment, the tubular rotator comprises a ramp surface arranged between the first and the second retaining member; wherein the counter retaining element is configured to move along the ramp surface from the first retaining member to the second retaining member.

According to one embodiment, the axial movement of the biased delivery member cover from the first retracted position to the first extended position causes the tubular rotator to rotate from the first rotation position to the second rotation position.

According to one embodiment, the medicament delivery device comprises a resilient member configured to proximally bias the biased delivery cover member.

According to one embodiment, the clicking ring is positioned between the tubular rotator and the medicament container along the longitudinal axis.

According to one embodiment, the clicking ring is positioned between the tubular rotator and the container carrier along the longitudinal axis.

According to one embodiment, the delivery member cover comprises a guide element; and the tubular rotator comprises a tubular body arranged with a guide track on its outer surface.

According to one embodiment, the guide element is configured to interact with the guide track such that an axial movement of the biased delivery member cover causes the tubular rotator to rotate such that the plunger rod is released from the tubular rotator.

According to one embodiment, the medicament delivery device can be an injection device, an on-body device, an inhalation device, a nasal sprayer or a medical sprayer.

According to one embodiment, the delivery member can be an injection needle, a catheter or a spray nozzle.

Other aspects, features, and advantages will be apparent from the summary above, as well as from the description that follows, including the figures and the claims.

Generally, all terms used in the claims are to be interpreted according to their ordinary meaning in the technical field, unless explicitly defined otherwise herein. All references to “a/an/the element, apparatus, component, means, etc. are to be interpreted openly as referring to at least one instance of the element, apparatus, component, means, etc.”, unless explicitly stated otherwise.

### BRIEF DESCRIPTION OF THE DRAWINGS

The specific embodiments of the inventive concept will now be described, by way of example, with reference to the accompanying drawings, in which:

FIG. 1 displays a medicament delivery device of the invention.

FIG. 2 displays an exploded view the medicament delivery device of FIG. 1 in detail.

FIG. 3 displays a perspective view of a tubular rotator.

FIG. 4 displays a perspective view of a plunger rod.

FIG. 5 displays a dose stop of the invention.

FIG. 6 displays an indication element of the invention.

FIG. 7 displays a cross-section view of the indication of FIG. 6.

FIG. 8 displays a perspective view of another embodiment of the indication element of the invention.

### DETAILED DESCRIPTION

The present application is directed to a feedback mechanism for a medicament delivery device and will now be described more fully hereinafter with reference to the accompanying drawings, in which exemplifying embodiments are shown. The feedback mechanism may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description.

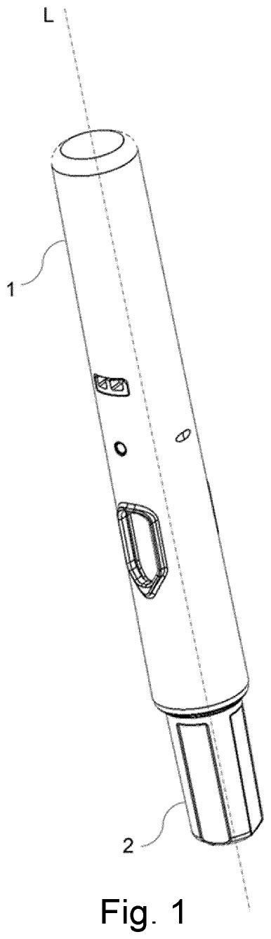

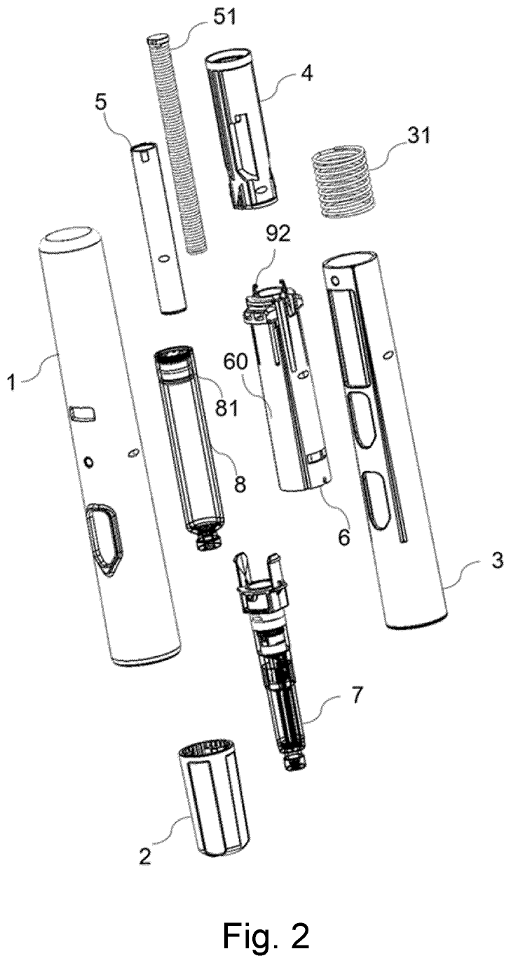

FIG. 1 illustrates a medicament delivery device in one embodiment of the invention with a housing 1 having a proximal end and a distal end along a longitudinal axis L; and a cap assembly 2 arranged on the proximal end of the housing 1 in an initial state of the medicament delivery device. As shown in FIG. 2, the medicament delivery device further comprises: a delivery member cover 3 axially movable but rotationally fixed in relation to the housing 1; a resilient member 31 arranged between the distal end of the delivery member cover 3 and an inner distal ledge of housing 1 and configured to bias the delivery member cover 3 in the proximal direction in relation to the housing 1; a tubular rotator 4. The tubular rotator 4 comprises a tubular body arranged within the housing 1 and is rotatable but axially immovable in relation to the housing 1. The medicament delivery device further comprises a plunger rod 5 axially movably arranged within the housing 1; a drive element 51 arranged within the plunger rod 5 and configured to bias the plunger rod in the proximal direction; a container carrier 6 arranged within the housing 1. The container carrier 6 is axially and rotationally fixed in relation to the housing 1 and configured to receive a medicament container 8. The medicament container 8 is configured to contain a medicament and comprises a stopper 81 arranged on its distal end and a pierceable membrane arranged on its proximal end.

In a preferred example, the medicament container is a container 8 configured be isolated from a medicament delivery member before use. In this example, the medicament delivery member needs to be fluidly communicated with the container 8 for delivering medicament, e.g., manually by a user or automatically before the medicament delivery starts. In this example, the medicament delivery device comprises a delivery member assembly 7 comprising a delivery member configured to deliver a dose of medicament to the end user during a medicament delivery operation. The delivery member assembly further comprises: a delivery member hub configured to hold the delivery member; an inner cap configured to cover the delivery member; and a retainer configured to threadedly engage with the inner cap in the initial state of the medicament delivery device. The cap assembly 2 is axially fixed to the inner cap of the delivery member assembly 7 and rotationally fixed with the inner cap in unidirectional rotation direction. The retainer is snap-fixed to the container carrier 6, such that the cap assembly 2 is attached to the housing 1 through the delivery member assembly 7 in the initial state of the medicament delivery device. The cap assembly 2 can be detached from the housing 1 of the medicament delivery device when the cap assembly 2 and the inner cap have been both screwed and disengaged from the retainer. Such screwing movement turns into an axial movement of the delivery member hub due to a coupling between the inner cap and the delivery member hub whereby the delivery member is moved together with the delivery member hub towards the medicament container 8 establishing a fluid communication with the medicament container 8. Alternatively, the medicament container is a syringe with an integral delivery member; in this example, the medicament delivery device does not comprise the delivery member assembly.

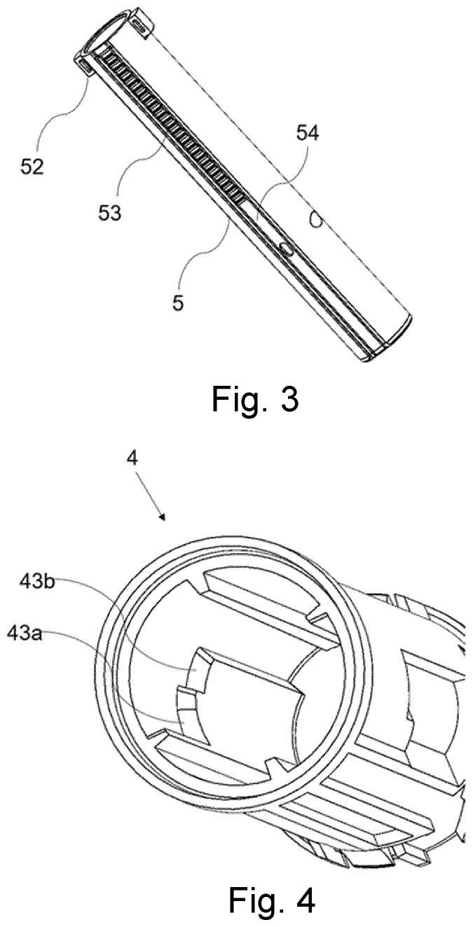

FIG. 3 illustrates the tubular rotator comprising a first retaining member 43a and a second retaining member 43b arranged on the inner surface of the tubular body. The first and the second retaining member is configured to selectively engage with the counter retaining member 52 on the plunger rod 5 as shown in FIG. 4, such that the proximal axial movement of the plunger rod is prevented. The counter retaining member 52 is configured to rest on the first retaining member 43a when the tubular rotator 4 is in a first rotation position; and is configured to disengage from the first retaining member 43a and moves proximally under the biasing force of the drive element 51 to engage with the second retaining member 43b when the tubular rotator 4 is in a second rotation position.

In one example, the plunger rod 5 is in its initial position when the counter retaining member 52 is engaged with the first retaining member 43a. When the plunger rod is in its initial position, the medicament delivery device is in its initial state wherein all components of the medicament delivery device have been assembled together and the device is ready to be delivered to an end user.

When the counter retaining member 52 of the plunger rod 5 is engaged with the second retaining member 43b of the tubular rotator 4, the plunger rod 5 is in a ready-to-be-used position, such that the medicament delivery device is now ready to carry out the medicament delivery operation.

It should be noted that, the medicament delivery device can optionally provide priming function, e.g., expelling air from the medicament container; mixing function, e.g., mixing multiple substances, and/or calibration function, e.g., fixing the start position of the plunger rod 5 relative to the housing before carrying out the medicament delivery operation. Alternatively, the medicament delivery device is designed without those functions, in this case, the first retaining member of the rotator is not necessarily. Furthermore, the rotator only needs to be rotated once, in other words, the rotator is rotatable from the second rotation position to a third rotation position.

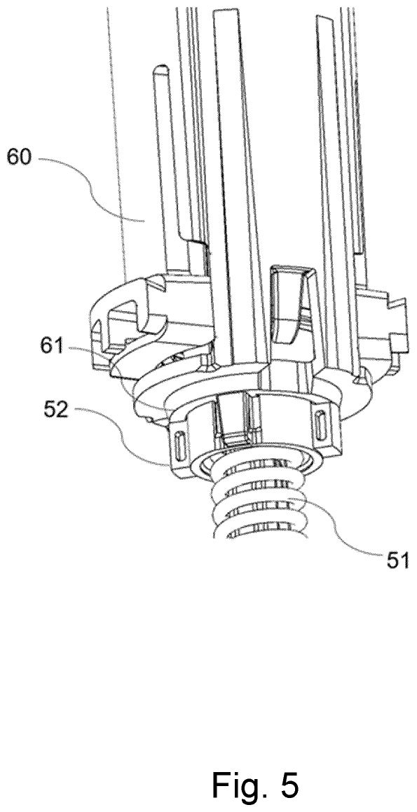

To perform the medicament delivery operation, the counter retaining member 52 of the plunger rod 5 is configured to disengage from the second retaining member 43b of the tubular rotator 4 when the tubular rotator 4 is in the third rotation position, such that the plunger rod is moved proximally under the biasing force of the drive element 51 and actuates on the stopper 81 to deliver a dose of medicament. The medicament delivery operation is therefore started. The counter retaining member 52 of the plunger rod 5 is configured to be proximally moved in order to engage with a dose stop 61 when the plunger rod 5 is in a final position, such that the medicament delivery operation is ended. The dose stop 61 can be a ledge arranged on the distal end of the container carrier 6 as shown in FIG. 5 or a ledge arranged on an extra component or a third retaining member arranged on the inner surface of the tubular rotator 4. Such that the delivered dose is determined by the distance between the second retaining member 43b and the dose stop which is also the travel distance of the plunger rod 5 from the ready-to-be-used position to a final position.

In a preferred example, as shown in FIGS. 2-4, the counter retaining member 52 of the plunger rod 5 is a protrusion extending from a body of the plunger rod 5; and the first and the second retaining members are ledges extending from an inner surface of the tubular rotator 4. Alternatively, the counter retaining member can be a recess/cut-out; and the retaining members of the tubular rotator can be flexible arms.

The tubular rotator 4 is configured to interact with the delivery member cover 3. In a preferred example, the delivery member cover 3 comprises a guide element 32 configured to move along a guide track 41 arranged on the outer surface of the tubular rotator 4. The interaction between the guide element 32 and the guide track 41 forces the tubular rotator 4 to rotate from the first to the second rotation position; and the second to the third rotation position.

The delivery member cover 3 can be manually pushed distally to its retracted position for triggering the medicament delivery operation. When the delivery member cover 3 moves to the retracted position, the guide element 32 is moved along a third inclined ledge 41g and an operation ledge 41h towards its second proximal distal end point of the guide track 41. When the guide element 32 passes the third inclined ledge 41g of the guide track 41, the tubular rotator 4 is rotated from the second rotation position to the third rotation position, such that the medicament delivery operation is started.

After the medicament delivery operation is ended, the delivery member cover 3 is no longer pushed to and retained on the second retraction position; the delivery member cover 3 is configured to move proximally to its second extended position under the biasing force of the resilient member 31 and configured to surround the delivery member again. The proximal movement of the delivery member cover 3 causes the guide element 32 proximally move along the operation ledge 41h of the guide track 41 to its second proximal end point 41i of the guide track 41. Before the guide element 32 moves into the second proximal end point 41i of the guide track 41, it passes a locking tongue 42 of the tubular rotator 4, such that the locking tongue 42 comprises a proximally directed cliff surface, therefore, once the guide element 32 moves into the second proximal end point 41i of the guide track 41, the further distal movement of the delivery member cover 3 is prevented due to the blocking between the guide element 32 and the proximally directed cliff surface of the locking tongue 42.

The delivery member protrudes from proximal end of the delivery member cover 3 when the delivery member cover 3 is in the first and the second retracted positions; and is covered by the delivery member cover 3 when the delivery member cover is in the first and the second extended positions.

The axial movement of the delivery member cover 3 driven by the resilient member 31 from the first retracted position to the first extended position causes the guide element 32 to move from the first distal end point 41c to the proximal end point 41f of the guide track 41. When the guide element 32 passes the first inclined ledge 41d and the second inclined ledge 41e, the tubular rotator 4 is gradually rotated from the first rotation position to the second rotation position.

It should be noted that, In one example where the rotator comprises the first retaining member 43a for initially holding the plunger rod 5, since the rotator 4 is engaged with the plunger rod 5 through the engagement between the first retaining member 43a and the counter retaining member 52, there will be a friction created between the first retaining member 43a and the counter retaining member 52 when the rotator 4 is rotating in relation to the plunger rod 5. The magnitude of the friction is depending on the accumulated force in the drive element 51, since it applies on the plunger rod 5, if the accumulated force in the drive element 51 is too high, the rotator 4 might not be rotated by the guide element 32 of the delivery member cover 3 which is proximally driven by the resilient member 31. Such a risk can be overcome by arranging a ramp surface to connect the first and the second retaining member. Therefore, the plunger rod 5 can move along the ramp surface from the first retaining member 43a to the second retaining member 43b. The rotation of the tubular rotator 4 from the first rotation position to the second rotation position is thereby mainly under the biasing force from the drive element 51.

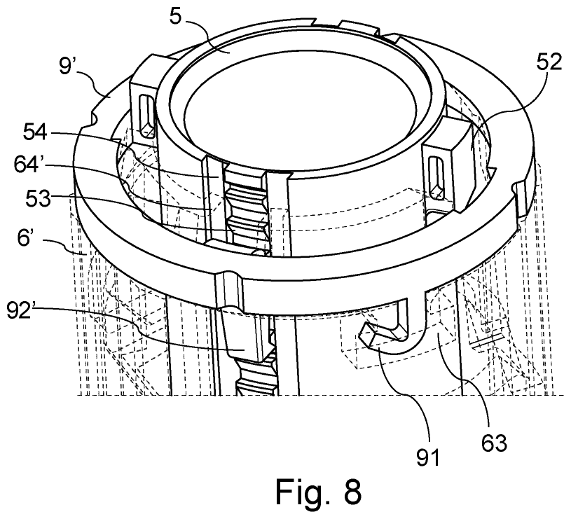

Furthermore, the medicament delivery device is configured to provide audible/ tactile feedback to the user of the medicament delivery device to indicate the progress of the medicament delivery operation. The feedback is provided by an interaction member 53 arranged on the plunger rod 5, preferably ratchet teeth extending in the direction of the longitudinal axis L; and a counter interaction member 92; 92′ adjacent to the distal end of the container 8. Once the plunger rod 5 proximally moves and passes the distal end of the medicament container 8, continuous feedback is thereby generated through the interaction between the interaction member 53 and the counter interaction member 92; 92′. In a preferred example, the counter interaction member 92; 92′ is a flexible arm with a free end sequentially engaging with each tooth of the ratchet teeth 53 when the interaction member 53 interacts the counter interaction member 92; 92′. In a preferred example, the plunger rod 5 comprises a longitudinally extending groove 54. In this example, the interaction member 53 is arranged within the groove 54. In one example where the interaction member 53 are ratchet teeth, the ratchet teeth 53 extends within the groove in the direction of the longitudinal axis L. In one example, as shown in FIG. 8, the container carrier 6′ comprises a ledge 64′ configured to be positioned within the groove 54 to guide the movement of the plunger rod 5; thus, the interaction member 53 is aligned with the counter interaction member 92.

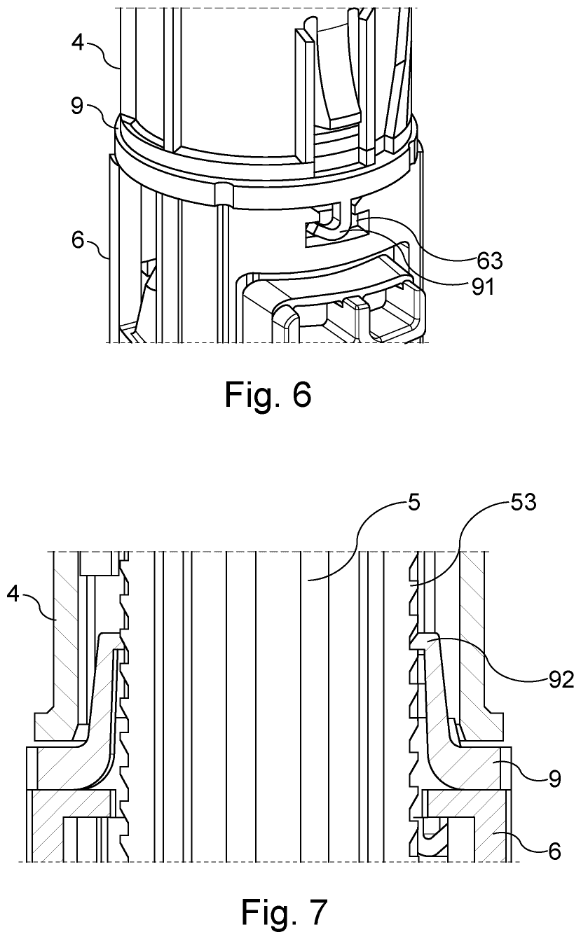

In one example, the medicament delivery device comprises a clicking ring 9; 9′, as shown in FIGS. 6-7. In one example, the clicking ring 9; 9′ is attached to the distal end of the container carrier 6; alternatively, the clicking ring is attached to the housing. In one example, the clicking ring 9; 9′ comprises a proximally extending hook 91, as shown in FIG. 6, in this example, the clicking ring 9; 9′ is attached to the container carrier 6 by engaging the hook 91 to a recess/cut-out 63 of the container carrier 6.

In one example where the counter interaction member 92; 92′ is a flexible arm, the free end of the flexible arm is directed towards the distal end of the housing, as shown in FIG. 7; alternatively, or additionally, the free end og the flexible arm is directed towards the proximal end of the housing, as shown in FIG. 8. In the former example, a louder sound indication may be generated; in the latter example, the continue indication may last longer.

As mentioned above, the feedback mechanism as described above is configured to provide audible/tactile feedback to the user to indicate the progress of the medicament delivery operation, However, the feedback is generally driven by the plunger rod. This can add friction to the injection and negatively affect injection time. Therefore, separating the clicking portion from other components, e.g., the container carrier or the housing into a separate clicking ring, enables more design flexibility. For example, the clicking ring can be made of different material than the housing or the container carrier; thus, material can be chosen to reduce friction.

In a preferred example, the clicking ring will be loosely assembled between the container carrier 6 and the tubular rotator 4. The audible/tactile feedback will be generated by the interaction member 53 of the plunger rod 5 and the counter interaction member 92; 92′ of the clicking ring 9; 9′; when the interaction between the plunger rod and the clicking ring causes the clicking ring to rattle, e.g., shaking, between the tubular rotator 4 and container carrier, as a result, louder audible feedback can be generated. In a preferred example, the clicking ring 9; 9′ is loosely attached to the container carrier 6, as shown in FIG. 6 and FIG. 8. In this example, the hook 91 is movable within the recess/cut-out 63 of the container carrier 6.

It should be noted that, in a preferred example, the clicking ring 9; 9′ is ring-shaped. Alternatively, the clicking ring is formed in a shaped of circlip, or comprises rectangular cross-section observed along the longitudinal axis L.

Furthermore, the housing of the medicament delivery device and/or the container carrier of the medicament delivery device, as mentioned in any example, may be provided with (i.e., molded in, molded with) a compound featuring persistently antimicrobial, antifungal, and/or antiviral properties. Alternatively, a compound featuring persistently antimicrobial, antifungal, and/or antiviral properties may be applied to the molded (i.e., finished) components through secondary processes (e.g., chemical vapor deposition), spraying, or dipping processes.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of medicaments that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary medicaments that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary medicaments that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary medicaments that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary medicaments that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Exemplary medicaments that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy medicaments include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A medicament delivery device, comprising:
a housing having a proximal end and a distal end;
a medicament container arranged within the housing;
a plunger rod movably arranged within the housing, wherein the plunger rod is configured to be moved into the medicament container so that contained medicament can be expelled, and wherein the plunger rod comprises an interaction member; and
a clicking ring arranged at a distal end of the medicament container,
wherein the counter interaction member of the clicking ring configured to interact with the interaction member of the plunger rod when the plunger rod is moved relative to the clicking ring such that audible/tactile feedback is generated to indicate a progress of expelling the medicament, and
wherein the clicking ring configured to rattle on the medicament container when the counter interaction member of the clicking ring interacts with the interaction member of the plunger rod.

17. The medicament delivery device according to claim 16, wherein the interaction member of the plunger rod is ratchet teeth extending in the direction of the longitudinal axis.

18. The medicament delivery device according to any of claims 17, wherein the counter interaction member of the clicking ring is a flexible arm.

19. The medicament delivery device according to claim 18, wherein the flexible arm comprising a free end configured to be in contact with the interaction member of the plunger rod, and wherein the free end points towards the proximal end of the housing.

20. The medicament delivery device according to claim 16, wherein the medicament delivery device comprises a container carrier, and wherein the medicament container is fixed within the container carrier.

21. The medicament delivery device according to claim 20, wherein the clicking ring is attached to a distal end of the container carrier.

22. The medicament delivery device according to claim 21, wherein the clicking ring is loosely attached to a distal end of the container carrier.

23. The medicament delivery device according to claim 21, wherein the clicking ring comprises a proximally directed hook configured to engage with a recess/cut-out of the container carrier such that the clicking ring is attached to the container carrier.

24. The medicament delivery device according to claim 23, wherein the hook is movable within the recess/cut-out.

25. The medicament delivery device according to claim 16, further comprising:
a delivery member cover arranged within the housing and is movable relative to the housing; and
a tubular rotator releasably engaged with the plunger rod, wherein the tubular rotator is engaged with the delivery member cover and is configured to be rotated by the delivery member cover to release the plunger rod.

26. The medicament delivery device according to claim 25, wherein the tubular rotator comprises a tubular body having at least one retaining member on its inner surface configured to releasably engaged with the plunger rod.

27. The medicament delivery device according to claim 25, wherein the clicking ring is positioned between the tubular rotator and the medicament container along the longitudinal axis.

28. The medicament delivery device according to claim 27, wherein the clicking ring is positioned between the tubular rotator and the container carrier along the longitudinal axis.

29. The medicament delivery device according to claim 25, wherein the delivery member cover comprises a guide element, wherein the tubular rotator comprises a tubular body arranged with a guide track on its outer surface, and wherein the guide element is configured to interact with the guide track such that an axial movement of the biased delivery member cover causes the tubular rotator to rotate such that the plunger rod is released from the tubular rotator.

30. The medicament delivery device according to claim 25, wherein the medicament delivery device comprises a resilient member is configured to proximally bias the delivery cover member.
